(19) United States
(12) Patent Application Publication
(10) Pub. No.: US 2020/0232963 A1
(43) Pub. Date: Jul. 23, 2020

(54) SYSTEM AND METHOD FOR AIRBORNE HYPERSPECTRAL DETECTION OF HYDROCARBON GAS LEAKS

(71) Applicant: Lawrence Livermore National Security, LLC, Livermore, CA (US)

(72) Inventor: Michael ZELINSKI, Oakland, CA (US)

(21) Appl. No.: 16/739,994

(22) Filed: Jan. 10, 2020

Related U.S. Application Data
(60) Provisional application No. 62/793,451, filed on Jan. 17, 2019.

(51) Int. Cl. G01N 33/22; G05D 1/10; G01N 21/3504; G05D 1/00; G01J 3/28

(52) U.S. Cl. CPC G01N 33/225; G05D 1/101; G01J 3/2823; G05D 1/0094; G01N 21/3504

(57) ABSTRACT

The present disclosure relates to a portable hydrocarbon plume monitoring system. The system is configured to be supportable at an elevated location above a region to be monitored for the presence of a hydrocarbon plume. The system has a housing and a hyperspectral sensor supported from the housing. The hyperspectral sensor images at least a portion of the region to be monitored and detects a presence of a hydrocarbon plume emanating from the region. The system also includes a computer for controlling operation of the hyperspectral sensor. The computer is adapted to use absorbance information and data generated by the hyperspectral sensor to determine the presence of a hydrocarbon plume emanating from the region.

### CROSS-REFERENCE TO RELATED APPLICATIONS

This application claims the benefit of U.S. Provisional Application No. 62/793,451, filed on Jan. 17, 2019. The entire disclosure of the above application is incorporated herein by reference.

### STATEMENT OF GOVERNMENT RIGHTS

The United States Government has rights in this invention pursuant to Contract No. DE-AC52-07NA27344 between the U.S. Department of Energy and Lawrence Livermore National Security, LLC, for the operation of Lawrence Livermore National Laboratory.

### FIELD

The present disclosure relates to methane detection systems and methods, and more particularly to a portable gas detection system adapted to be removably secured to an airborne vehicle which is able to remotely detect hydrocarbon gas leaks while flying over selected geographical areas.

### BACKGROUND

The statements in this section merely provide background information related to the present disclosure and may not constitute prior art.

It is well known that methane gas is a highly potent greenhouse gas. By some estimates, methane is believed to have 20-80 times the heat trapping capacity that CO2 possesses. While methane gas is a byproduct of many natural phenomena, the levels related to anthropogenic releases are rising substantially.

Methane leaks can occur in various types of infrastructure used in a number of different industries. However, the largest contributions of methane gas leakage likely come from oil/gas production infrastructure, from agriculture (e.g., beef production), and from waste landfills. In many cases methane gas leaks occurring in these industries are substantial and go unnoticed. As a result, undetected methane gas leaks may form a significant safety concern as well as an environmental concern. In cases such as the San Bruno methane gas pipeline explosion, which occurred in San Bruno, Calif. on Sep. 9, 2010, significant property damage and multiple fatalities occurred when a 30 inch (76 cm) diameter natural gas pipeline exploded.

In response to the dangers of fugitive methane gas leaks, some states (e.g., California) have enacted laws mandating the monitoring of methane hotspots. As anthropogenic levels rise, and natural gas technology proliferates, it is expected that more states (and possibly nations other than the United States) will begin monitoring various locations and infrastructure for the unintended release of methane gas.

In addition to increasing government mandating of methane monitoring, it is expected that various entities and industries will become more amenable to undertaking their own methane gas monitoring efforts, and voluntarily implement and/or contract for monitoring services to ensure they are in compliance with government mandated monitoring regulations. Investments have already been made by oil and gas companies on projects such as the MethaneSAT, which involves the use of a small satellite to monitor methane emissions from human activities. There are numerous other startup companies that are also being funded by the oil and gas industry with the goal of developing technologies for more cost effective methane monitoring and detection.

The inventor of the subject matter of the present disclosure is aware of three specific technologies/approaches that are able to achieve the goal of methane hotspot detection and localization. The first is a chemical sniffer known as a “Picarro sensor” (https://www.picarro.com/) available from Picarro, Inc. of Santa Clara, Calif. This sensor is a point sensor; however, it cannot perform remote methane hotspot detection. Furthermore, a Picarro sensor is not capable of “imaging” a relatively large area to efficiently determine the specific location of a leak within the imaged area.

The second technology involves a helicopter mounted laser-based gas detection technology, such as offered by LaSen, Inc. (www.lasen.com). This type of system uses an airborne laser tuned to detect one specific hydrocarbon, which is carried on a helicopter. The laser images a relatively small area as the helicopter is in flight, and flying typically at a relatively slow speed (e.g., about 60 mph or 100 kmh). Such systems are capable, but generally are quite expensive to operate and relatively slow (e.g., often around $17 k/hr at 50 mph). Both the Picarro sensor and helicopter carried laser detection systems are technologies which are widely used in present day industry.

The third known approach is presently being developed by a number of small entities, and aims to monitor methane gas emissions from outer space. Some of these companies plan to use a similar SWIR hyperspectral sensor as the one suggested here, however acquiring useful results from a low Earth orbit (“LEO”) spacecraft is expected to be extremely challenging, for a number of reasons. One specific reason is the likelihood that an LEO based spacecraft will likely need to employ a relatively large (and costly) telescope.

It will also be appreciated that while airborne monitoring of hydrocarbon plumes emanating from pipelines may be done with an instrument having a relatively narrow field of view, the monitoring of oil and gas fields presents significant additional challenges with present day monitoring/detection technologies. Oil and gas fields may span many square miles. As such, airborne instruments with a narrow field of view are not ideal for cost effective monitoring of such large areas. Ideally, one would need a system that can be deployed from an airborne platform which can image larges areas, possibly encompassing several hundred square meters or more, for the system to be operated cost effectively. This is not possible with present day detection systems and methods.

Accordingly, a need still exists for a most cost effective and easily implemented system and method for enabling rapid detection of methane gas leaks over long distances, such as those spanned by pipelines, or over relatively large areas, such as those spanned by oil/gas fields.

### SUMMARY

This section provides a general summary of the disclosure, and is not a comprehensive disclosure of its full scope or all of its features.

In one aspect the present disclosure relates to a portable hydrocarbon plume monitoring system configured to be supportable at an elevated location above a region to be monitored for the presence of a hydrocarbon plume. The system may comprise a housing and a hyperspectral sensor supported from the housing. The hyperspectral sensor images at least a portion of the region to be monitored and detects a presence of a hydrocarbon plume emanating from the region. The system may also include a computer for controlling operation of the hyperspectral sensor. The computer may use absorbance information and data generated by the hyperspectral sensor to determine the presence of a hydrocarbon plume emanating from the region.

In another aspect the present disclosure relates to a portable hydrocarbon plume monitoring system configured to be installed on an aircraft.

The system may comprise a housing having an opening, and a hyperspectral sensor contained in the housing and positioned over the opening. The opening may be positioned over an additional opening in a body portion of the aircraft when the system is installed on the aircraft for use. The system enables the hyperspectral sensor to image a predetermined swath of ground surface below the aircraft when the aircraft is in flight traversing a region to be monitored. The system may include a computer for controlling operation of the hyperspectral sensor; a global positioning system (GPS) unit in communication with the computer; a memory for storing an algorithm for use by the computer in analyzing data obtained by the hyperspectral sensor as the aircraft is in flight traversing the region; and a database accessible by the computer for containing absorbance information pertaining to different types of hydrocarbon gasses. The computer uses the absorbance information and data from the hyperspectral sensor while the aircraft is in flight over the region to determine the presence of a hydrocarbon plume. The computer may record GPS data from the GPS unit which is associated with the latitude and longitude of each detected hydrocarbon plume.

In still another aspect the present disclosure relates to a method for monitoring for and detecting the presence of a hydrocarbon plume in a region. The method may comprise supporting a hyperspectral sensor from a housing, where the housing is supported from a portion of an aircraft, and the hyperspectral sensor is aimed at a ground surface during flight of the aircraft. The method may further include using the hyperspectral sensor to monitor the ground surface for a hydrocarbon plume as the aircraft is in flight over the region. The method may further include using a computer housed within the housing to communicate with the hyperspectral sensor and to use stored absorbance information to help analyze data being generated by the hyperspectral sensor as the aircraft is in flight, to determine the presence of a hydrocarbon plume emanating from the region as the aircraft flies over the region. The method may further include using the computer to note a location of a detected hydrocarbon plume.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The drawings described herein are for illustration purposes only and are not intended to limit the scope of the present disclosure in any way.

FIG. 1 is a high level block diagram of one embodiment of a portable system adapted for use on airborne vehicles, to perform hyperspectral methane sensing, in accordance with the present disclosure; and

FIG. 2 is a high level flowchart illustrating various operations that may be performed by the system of FIG. 1 to obtain and record hyperspectral data while using an airborne vehicle.

### DETAILED DESCRIPTION

The following description is merely exemplary in nature and is not intended to limit the present disclosure, application, or uses. It should be understood that throughout the drawings, corresponding reference numerals indicate like or corresponding parts and features.

It is expected that any form of new technology for hydrocarbon gas monitoring will need to be cost effective, while also being easily implemented on present day vehicles, and also while enabling entities to meet present day regulatory requirements. The present disclosure discloses systems and methods which can be deployed effectively from a small, relatively inexpensively operated, fixed wing aircraft to image relatively large areas, and quickly detect the presence of a hydrocarbon gas emanating from an underground structure or an above ground structure.

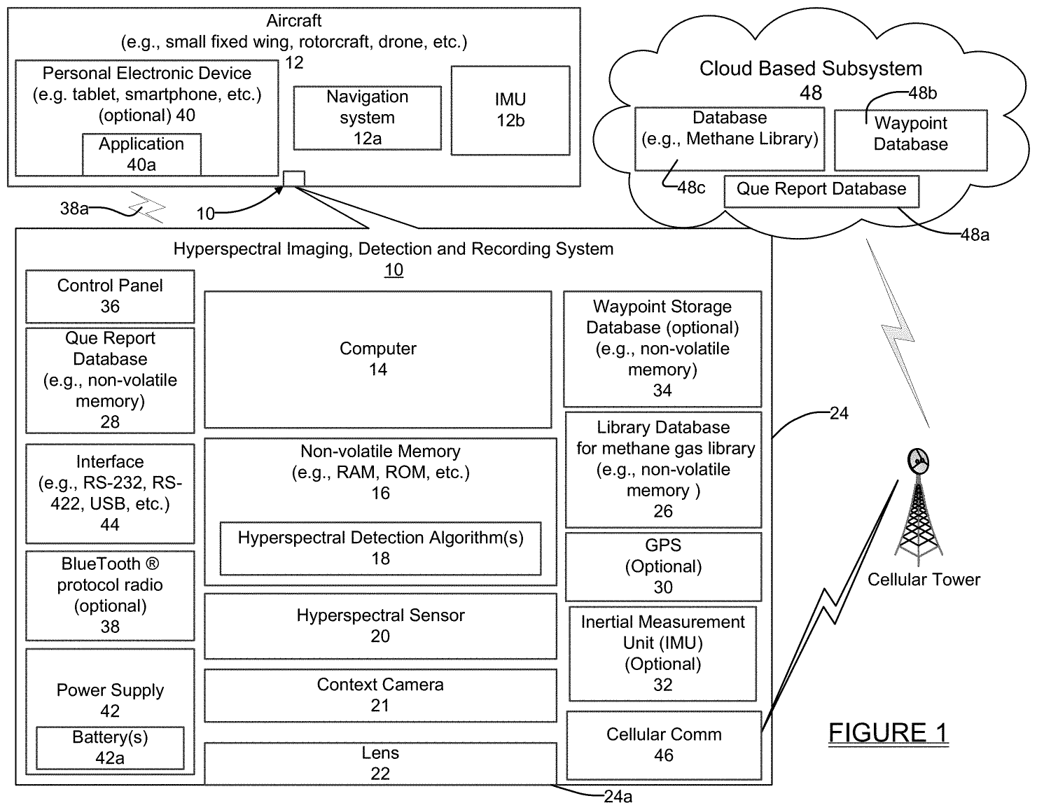

Referring to FIG. 1 a system 10 in accordance with the present disclosure is shown for remotely detecting a hydrocarbon gas plume from an airborne platform. In this example the system 10 may be employed from a small, fixed wing, piston engine-driven propeller aircraft or turbo-propeller driven aircraft 12. Such aircraft are widely available from various companies, for example Textron Aviation Services (i.e., Cessna, Beechcraft, Hawker) and other aviation companies, and are relatively inexpensive to operate (generally as little as about $120/hr-$200/hr) when leased. However, it will be appreciated that the system 10 may be employed with virtually any airborne mobile platform, and as such may find utility with rotorcraft, unmanned aerial vehicles (e.g., drones), and possibly even with land vehicles. Possible implementations may even include mounting the system 10 in a stationary position on a tower on a natural gas tanker ship and using the system to monitor for any natural gas plume that may be leaking from the ship. Also, the system 10 may be used at a stationary location, for example at the top of a ground based tower overlooking a designated area, to monitor a large designated area. Still further, the system 10 may potentially be employed in connection with a balloon anchored by one or more guy files such that it remains at a designated elevation. The system 10 may be secured to structure associated with the balloon such that it looks downwardly toward the ground surface to image a predetermined area on the ground surface. In the balloon or tower mounted applications, it may be helpful to incorporate a subsystem that scans the system's 10 field of view to image even larger areas. Still further, a network of tower mounted or balloon supported systems 10 could be used to monitor a relatively large contiguous or non-contiguous swath of ground area. Still further, the system 10 may be located on a ground vehicle (e.g., truck), and possibly on a vertical mast carried by the ground vehicle, and used to image a designated area as the truck is driven along (or back and forth) over the designated area. Merely for convenience, the vehicle which the system 10 is employed on in the following discussion will be referred to as the “aircraft 12”.

The system 10 forms a hyperspectral imaging detection and recording system which is able to detect the presence of a hydrocarbon gas plume in those areas being imaged by the system 10 as the aircraft flies along a given route. The system 10 may include a computer 14, a non-volatile memory 16 which stores one or more hyperspectral detection algorithms 18, a hyperspectral sensor 20, a context camera 21, and an optically transparent lens 22 (e.g., polycarbonate lens). The lens 22 may be positioned in an opening 24a along a bottom wall of a housing 24. The hyperspectral sensor 20 is a commercially available component. The context camera 21 (e.g., a high resolution DSLR) may be mounted within the housing 24 to image approximately the same area as the hyperspectral sensor 20 and to collect high resolution images, possibly at a rate of about 0.5 Hz-2 Hz, together with GPS data for each image, as the aircraft 12 flies along a predetermined route. The lens 22 enables a clear line of sight for the hyperspectral sensor 20 and the context camera 21 toward a ground surface that the aircraft 12 is flying over. The housing 24 may be a metal or plastic structure, or may be made from other suitably strong materials, and may be secured using any suitable fasteners or other means that secure the system 10 against movement once mounted in the aircraft 12.

In one example the computer 14 may comprise a Jetson AGX Xavier computer available from NVIDIA Corporation of Santa Clara, Calif., which is a powerful, compact and air-cooled computer. However, any computer having computing capability capable of analyzing the data provided by the hyperspectral sensor 20 may be used. The hyperspectral sensor 20 may comprise a short-wave infrared (SWIR) hyperspectral sensor which is commercially available from Corning Incorporated of Corning, N.Y. The hyperspectral detection algorithm(s) 18 compare(s) obtained data (i.e., sensed hyperspectral data) from the hyperspectral sensor 20 to information contained in a hydrocarbon gas library database 26 stored in non-volatile memory (e.g., RAM or ROM). More specifically, the library database 26 contains absorbance data/values for each wavelength in the infrared spectrum. The absorbance data covering the gas spectrum for at least methane gas is presently available from Pacific Northwest National Lab (Richland, Wash., and associated with the U.S. Department of Energy) for commercial use.

By using the hyperspectral detection algorithm(s) 18 and the computer 14 to compare obtained hyperspectral data to the data available in the library database 26, the system 10 is able to suppress and/or filter out background noise and detect when hydrocarbon gas is present within an area being imaged by the hyperspectral sensor 20. As will be appreciated, the presence of hydrocarbon gas will result in the gas absorbing solar photons that are being reflected from the ground back up toward the aircraft 12. This occurrence is detected by the hyperspectral sensor 22. Comparing readings obtained by the hyperspectral sensor 20 with the data stored in the library database 26 enables the presence of a specific type of hydrocarbon gas (e.g., methane) to be detected in the sensed data. More specifically, it will be appreciated that the hyperspectral sensor 20 houses a focal plane array (FPA), which is a light sensitive semiconductor. When photons of a specific band pass hit the FPA, a percentage of these photons create electrons, which are subsequently converted into a voltage, and then to a digital number by an analog-to-digital (A/D) converter which the computer 14 uses. And while the system 10 is expected to find particularly utility in detecting plumes of methane gas, it will be appreciated that the system 10 may be “tuned” to other narrow ranges of the non-visible frequency spectrum, to enable the detection of propane, butane and other types of hydrocarbon gasses besides methane. This may involve adding absorbance information for other specific types of hydrocarbons to the library database 26, and possibly other minor modifications to the system 10.

When hydrocarbon gas is detected by the system 10, data may be stored in a que report database 28, which is also contained in non-volatile memory (e.g., RAM or ROM). The stored data may include time, latitude, longitude and aircraft elevation data provided by an on-board global positioning system (GPS) 30 incorporated into the system 10. Recorded data may also include inertial measurement data from an inertial measurement unit (IMU) 32 incorporated into the system 10, which records the IMU data along with the data generated by the hyperspectral sensor 20. The IMU 32 may provide real time data such as roll, pitch and yaw data, which provides the real time orientation of the system 10 at every instant that an image is obtained and recorded using the hyperspectral sensor 20. The IMU 32 data may be highly valuable in enabling highly accurate identification of the precise location on the ground surface where a hydrocarbon plume is emanating from. It will be appreciated, however, that the GPS 30 and the IMU 32 may be optional components; if a hardwired connection is established with the aircraft's 12 navigation system through a suitable interface or cable, then the aircraft's on-board GPS and IMU may be used to provide the GPS and IMU data to the system 10 as measurements are taken by the system 10, and then stored along with data generated by the system 10.

The system 10 may be configured such that the hyperspectral sensor 20 obtains measurements at a predetermined sampling frequency. In one example, that frequency may typically be about 10 Hz-1000 Hz, although operation of the system 10 at higher or lower frequencies is also possible. The selected sampling frequency may depend in part on what speed the aircraft 12 is being flown at, with higher aircraft speeds generally using a higher sampling frequency. The system 10 is expected to be used at flight altitudes of typically between 1500 feet-10,000 feet, although higher or lower altitudes of operation are possible. Generally, speaking, the sensitivity of the system 10 will be increased when flying at lower altitudes closer to 1500 feet, although the hyperspectral sensor 20 will be imaging a smaller area, as compared to when the aircraft 12 is flying at closer to (or at) 10,000 feet. Accordingly, it is expected that the sensitivity of the system 10 to detect extremely small gas plumes will be optimized when flying at close to, or even below, an altitude of 1500 feet.

The system 10 may optionally include a waypoint storage database 34 (i.e., non-volatile memory such as RAM or ROM) for storing a collection of waypoints that define a predetermined route that the aircraft 12 is to travel during operation. This may be useful for situations where the aircraft 12 is to follow a pipeline that is only partially visible from the air, or otherwise not visible at all from the air. If a hardwired connection is made to the aircraft's 12 on-board NAV system, then this waypoint information may be loaded automatically or via a user entered command from a suitable control panel 36 on the housing 24 of the system 10 after the system 10 is powered up.

With further reference to FIG. 1, the system 10 may optionally include a BLUETOOTH® protocol radio 36 which enables a short range wireless connection link 38a to be made with a personal electronic device (e.g., smartphone, tablet, laptop, etc.) 40 carried by the aircraft's pilot or by another individual who is setting up the system 10 for operation in the aircraft 12. If this configuration is chosen, then a suitable application 40a may be stored on the personal electronic device 40a which enables at least partial control over the computer 14, as well as selections and commands to be input by the user at the personal electronic device 40. The short range wireless connection may also enable the waypoint information stored in the waypoint database 34 to be accessed by the aircraft's pilot or another individual setting up the system 10 on the aircraft, and then manually input into the aircraft's NAV system 12a. And as noted above, an existing inertial measurement unit (IMU) 12b on the aircraft 12 may provide IMU data to the system 10 if a communications link is established between the aircraft's on-board communications system and the system 10.

With continuing reference to FIG. 1, the system 10 may also include a suitable power supply 42, for example a DC power supply powered by one or more on-board DC batteries 42a. Alternatively, it may be possible to obtain DC power from the aircraft's electrical system, and both implementations are envisioned by the present disclosure. Optionally, a communications interface 44 (e.g., RS-232; RS-422; USB, FIREWIRE® protocol, or parallel interface, etc.) may be included to enable coupling of the system 10 to an external computer, laptop or other electronic instrumentation for bi-directional communication with the external device. In this regard it will be appreciated that a suitable powerful laptop computer loaded with the hyperspectral detection algorithm(s) 18 may be used to perform the processing/analysis of the data collected by the hyperspectral sensor 20, rather than the dedicated computer 14. In this event, the laptop computer may also be used to communicate with a remote (e.g., cloud-based) subsystem to facilitate transfer of the collected data (either in real time or after the aircraft 12 has landed) to one or more cloud-based subsystem for analysis and/or storage.

Further options for using the system 10 may include a cellular communications subsystem 46 for enabling the system 10 to communicate during flight with a wireless cellular network (e.g., 3G, 4G, etc.), and to access the Internet via the wireless cellular network to communicate with a cloud-based subsystem 48. With this configuration, the system 10 may be able to upload recorded data in real time to a cloud-based que database 48a, or to obtain waypoints from a cloud-based waypoint database 48b, or optionally to obtain information from a library database 48c containing absorbance values for different types of hydrocarbon gasses (i.e., the same or similar as library database 26). However, it may be preferred to maintain the database of important absorbance information on the library database 26 to optimize use of, and access to, such data by the computer 14. Optionally, a satellite communications system may be used to obtain the recorded data in real time from the system 10.

The system 10 may be physically secured over a suitable sized opening (not shown) in the fuselage of the aircraft 12 which permits an unobstructed view of the hyperspectral sensor 20 through the lens 22, or possibly even on an exterior surface (e.g., wing nose cone or tail) of the aircraft. The opening to accommodate the lens 22 need not be a large opening, and an opening of typically about 5 inches×5 inches, or a generally circular opening having a diameter of about 5 inches, will suffice. The system 10 is highly compact and the housing 24 may have a size comparable to a small shoebox, ranging up to a small briefcase, with an easily hand-carryable weight in the range of about 5 lbs-20 lbs, and more typically about 10 lbs or less. As such, the system 10 may be easily and cost effectively shipped to a designated airport and easily hand carried on-board the aircraft 12 by an individual.

In another implementation the system 10 may be housed in a suitable “pod” adapted and approved to be secured to an exterior surface of an aircraft, for example to a wing, support strut, tail, fuselage, etc., such that it has a field of view below the aircraft. In one example the pod may be a commercial pod available from SkylMD, Inc. of Richmond, Calif., which is presently certified for flight on some small fixed-wing aircraft. In this implementation, the system 10 may have a size similar to a soccer ball. Alternatively, the system 10 may be permanently installed in the aircraft 12. If the aircraft 12 is a relatively small drone, then the aircraft will be controlled remotely as well. In either event, the system 10 has a small footprint and does not require significant modifications to the aircraft 12 or require significant space within the aircraft, or add significant weight to the aircraft, or require significant additional modifications to the aircraft 12 or otherwise significantly affect the payload of the aircraft.

During operation, as noted above, the aircraft 12 may be flown from one waypoint to the next to ensure that the aircraft flies over a designated route or path which lies above the ground areas that one wishes to observe. Alternatively, the aircraft 12 may be flown in a back-and forth pattern, similar to a raster scan pattern, over a designated area to image the area. At any particular location where it is suspected that a hydrocarbon plume is more likely to exist, the aircraft 12 may be flown at a lower elevation to even further increase the detection sensitivity of the system 10.

Regardless of the path or route that the aircraft 12 flies, small fixed wing, propeller-driven aircraft such as mentioned above can easily cruise at 100 mph, which is still about twice the speed of many present day rotorcraft. Small fixed-wing aircraft are also considerably less expensive to lease and operate (per hour) than a rotorcraft (i.e., helicopter). Accordingly, the system 10 is expected to enable a significant cost savings in carrying out airborne detection of gas plumes, as opposed to what would be possible with present day rotorcraft-based systems.

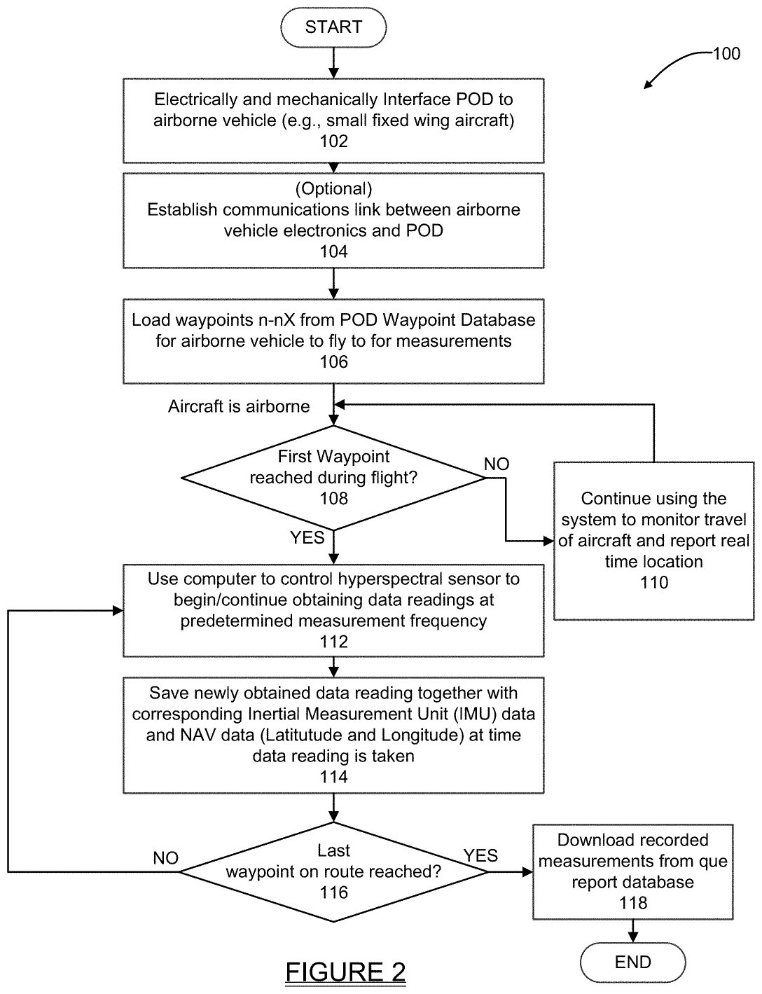

Referring to FIG. 2, a high level flowchart 100 is shown that sets forth example operations that may be performed by the system 10 of FIG. 1. At operation 102 the system 10 may be mechanically affixed such that the lens 22 is positioned over the opening in the fuselage of the aircraft 12, or otherwise on some exterior surface of the aircraft facing generally downwardly. Optionally, the system 10 may be electronically coupled via a suitable communications cable to the aircraft's 12 on-board electronics system as well, as indicated at operation 104.

At operation 106 waypoints n through nX from the waypoint storage database 34 may be loaded into the aircraft's 12 navigation system 12a. The waypoints may be loaded manually by an individual (e.g., pilot) or automatically if the system 10 is electronically coupled to the aircraft's electronics. Once the aircraft 12 becomes airborne the system 10 may begin checking for when the first, waypoint is reached, as indicated at operation 108, either by using the GPS system 30 or from information supplied by the aircraft's navigation system 12a. If the first waypoint is not detected, then operation 108 is repeated, for example every 0.5 seconds-5 seconds, until the check at operation 108 indicates that the first waypoint has been reached.

At the first waypoint the computer 14 turns on the hyperspectral sensor 20 to begin collecting measurements and comparing each measurement to predetermined data from the library database 26, using the hyperspectral detection algorithm(s) 18. At operation 114, the presence of any detected hydrocarbon plume will be recorded in the que report database 28 and/or otherwise reported to a remote cloud-based database for storage, as described above. Importantly, the GPS and IMU data associated with the detection of a hydrocarbon plume is recorded in real time in the que report database 28 to enable high resolution identification of the precise location of the plume on the ground surface. In this regard it will be appreciated that the data of the IMU 32 may be particularly helpful to account for instances where the aircraft is banking slightly while a measurement is being taken by the system 10, or is experiencing roll, pitch or yaw movements which affect aiming of the hyperspectral sensor 20. Using the information from the IMU 32 significantly enhances the accuracy in identifying the exact longitude and latitude coordinates of hydrocarbon plumes emanating from the ground surface.

At operation 116 the computer 14 checks if the last waypoint on the route has been reached. If this check produces a “No” answer, then operations 112 and 114 may be repeated. If the answer is “Yes”, then the measurement process may be ended and the computer 14 turns off the hyperspectral sensor 20. At operation 118, recorded measurements obtained by the system 10 during the flight may be downloaded from the que report database 28 immediately or at some later time. It is also possible that if a cellular or satellite communications link is employed, that obtained measurements from the system 10 may be uploaded in real time to the cloud-based subsystem 48 as the aircraft 12 is in flight flying from one waypoint to the next. Optionally, if a short range wireless connection exists with the user's personal electronic device 40, then measurement data may be uploaded in real time to the user's device as well, or in the alternative.

The system 10, due to its compact, lightweight configuration, as well as its ability to be carried on a small fixed wing aircraft, enables hydrocarbon plume detection to be carried out in a highly cost effective manner, and considerably more cost effectively than what is possible with present day airborne systems that are carried on rotorcraft. Because the system 10 is able to be used on a small, fixed-wing aircraft, which typically fly at considerably greater speeds than rotorcraft, larger areas may be imaged in a significantly shorter period of time than what would be possible with rotorcraft based hydrocarbon plume detection system. The portability of the system 10 enables it to be easily shipped to a designated airport or field location, and hand carried by an individual onto an aircraft for installation, or even optionally permanently installed in a small fixed wing aircraft dedicated to hydrocarbon plume monitoring.

While various embodiments have been described, those skilled in the art will recognize modifications or variations which might be made without departing from the present disclosure. The examples illustrate the various embodiments and are not intended to limit the present disclosure. Therefore, the description and claims should be interpreted liberally with only such limitation as is necessary in view of the pertinent prior art.

What is claimed is:

1. A portable hydrocarbon plume monitoring system configured to be supportable at an elevated location above a region to be monitored for the presence of a hydrocarbon plume, the system comprising:
a housing;
a hyperspectral sensor supported from the housing which images at least a portion of the region to be monitored, and which is configured to detect a presence of a hydrocarbon plume emanating from the at least a portion of the region;
a computer for controlling operation of the hyperspectral sensor; and
the computer further adapted to use absorbance information and data generated by the hyperspectral sensor to determine the presence of a hydrocarbon plume emanating from the region.

2. The system of claim 1, further comprising:
a camera supported from the housing and configured to produce images of the region being imaged by the hyperspectral sensor.

3. The system of claim 1, wherein the housing is configured to be mounted on an airborne platform.

4. The system of claim 1, wherein the housing is configured to be mounted on a tower.

5. The system of claim 3, wherein:
the housing includes an opening; and
the hyperspectral sensor contained is positioned over the opening, the opening being positioned over an additional opening in a body portion of the airborne platform when the system is installed on the airborne platform for use, to enable the hyperspectral sensor to image a predetermined swath of ground surface, which forms a portion of the region, below the airborne platform when the airborne platform is in use and moving over the region.

6. The system of claim 1, further comprising a global positioning system (GPS) unit in communication with the computer, for determining a longitude and latitude of the system when a hydrocarbon plume is detected, which corresponds to a longitude and latitude of the detected hydrocarbon plume, and providing information corresponding to the longitude and latitude of the system to the computer.

7. The system of claim 1, further comprising a memory in communication with the computer for storing an algorithm for use by the computer in analyzing data obtained by the hyperspectral sensor.

8. The system of claim 1, further comprising a database accessible by the computer for containing absorbance information pertaining to different types of hydrocarbon gasses.

9. The system of claim 1, wherein the system is carried on an airborne platform, and wherein the system further comprises a que report database, the que report database in communication with the computer and configured to store data relating to a detected hydrocarbon plume including at least one of time, latitude, longitude, elevation, global positioning data and inertial measurement data.

10. The system of claim 1, wherein the hyperspectral sensor obtains measurements of the region at a predetermined sampling frequency.

11. The system of claim 10, wherein the predetermined sampling frequency comprises a frequency in a range from 10Hz to 1000Hz.

12. The system of claim 1, wherein the system is carried on an airborne mobile platform, and wherein the system further comprises a waypoint storage database for storing a collection of waypoints that define a predetermined route that the airborne mobile platform is to travel in monitoring for a hydrocarbon plume.

13. The system of claim 1, further comprising a short range wireless radio in communication with the computer for enabling a short range wireless connection link to be made with a personal electronic device.

14. The system of claim 1, wherein the system includes a cellular communications link for enabling the system to communicate with a wireless cellular network during operation of the system.

15. The system of claim 8, further comprising a global positioning system (GPS) unit in communication with the computer, for determining a longitude and latitude of the system when a hydrocarbon plume is detected; and
wherein the computer is configured to use using the absorbance information and the hyperspectral sensor while system is moving across the region to determine the presence of a hydrocarbon plume, and to record GPS data from the GPS unit which is associated with the latitude and longitude of each detected said hydrocarbon plume.

16. The system of claim 1, wherein the hyperspectral sensor comprises a short wave, infrared hyperspectral sensor.

17. A portable hydrocarbon plume monitoring system configured to be installed on an aircraft, the system comprising:
a housing having an opening;
a hyperspectral sensor contained in the housing and positioned over the opening, the opening being positioned over an additional opening in a body portion of the aircraft when the system is installed on the aircraft for use, to enable the hyperspectral sensor to image a predetermined swath of ground surface below the aircraft when the aircraft is in flight traversing a region to be monitored;
a computer for controlling operation of the hyperspectral sensor;
a global positioning system (GPS) unit in communication with the computer;
a memory for storing an algorithm for use by the computer in analyzing data obtained by the hyperspectral sensor as the aircraft is in flight traversing the region;
a database accessible by the computer for containing absorbance information pertaining to different types of hydrocarbon gasses; and
the computer using the absorbance information and the hyperspectral sensor while the aircraft is in flight over the region to determine the presence of a hydrocarbon plume, and to record GPS data from the GPS unit which is associated with the latitude and longitude of each detected said hydrocarbon plume.

18. The system of claim 17, further comprising a context camera supported from the housing, and configured to image an area similar to that being imaged by the hyperspectral sensor and to periodically obtain camera images of the region being traversed, to enable at least one of the camera images to be correlated to an area of the region where a hydrocarbon plume is detected.

19. A method for monitoring for and detecting the presence of a hydrocarbon plume in a region, the method comprising:
supporting a hyperspectral sensor from a housing, where the housing is supported from a portion of an aircraft, and the hyperspectral sensor is aimed at a ground surface during flight of the aircraft;
using the hyperspectral sensor to monitor the ground surface for a hydrocarbon plume as the aircraft is in flight over the region;
using a computer housed within the housing to communicate with the hyperspectral sensor and to use stored absorbance information to help analyze data being generated by the hyperspectral sensor as the aircraft is in flight, to determine the presence of a hydrocarbon plume emanating from the region; and
using the computer to note a location of a detected hydrocarbon plume.

20. The method of claim 19, further comprising using a camera supported from the housing to obtain images of a ground surface of the region as the aircraft is in flight, and to provide an image of an area of the ground surface corresponding to where a hydrocarbon plume has been detected.
